(19) United States
(12) Patent Application Publication
(10) Pub. No.: US 2003/0083571 A1
(43) Pub. Date: May 1, 2003

(54) 0-Order phase detecting method and MRI system

(76) Inventor: Yoshikazu Ikezaki, Tokyo (JP)

Correspondence Address: Patrick W. Rasche, Armstrong Teasdale LLP, Suite 2600, One Metropolitan Sq., St. Louis, MO, 63102, US

(21) Appl. No.: 10/279,803

(22) Filed: Oct. 24, 2002

(30) Foreign Application Priority Data
Oct. 26, 2001 (JP) 2001-328447

(51) Int. Cl. A61B 5/05

(52) U.S. Cl. 600/410

(57) ABSTRACT

The present invention is intended to detect a 0-order phase that correctly represents the 0-order phase of an MR signal. The phase of a composite vector calculated using the complex vectors at all sampling points that result from Fourier transform of an MR signal is adopted as a 0-order phase.

### BACKGROUND OF THE INVENTION

The present invention relates to a 0-order phase detecting method and a magnetic resonance imaging (MRI) system. More particularly, the present invention is concerned with a 0-order phase detecting method and an MRI system capable of correctly detecting a 0-order phase of an MR signal.

FIG. 5 shows a primary example of an imaging pulse sequence adapted to a multi-shot diffusion enhancement echo planar imaging (EPI) method.

According to the imaging pulse sequence, an excitation pulse RF90 and a slice-selective magnetic field gradient SG90 are applied. Thereafter, a motion probing gradient (MPG) pulse MPG is applied. An inversion RF pulse RF180 and an inversion slice-selective magnetic field gradient SG180 are then applied. An MPG pulse MPG is then applied. Thereafter, a phase-encoding magnetic field gradient pdn is applied. Pulses r1, etc., and rm of a data acquisition readout magnetic field gradient that alternately reverses in polarity are applied consecutively. Moreover, pulses p2, etc., and pM of a phase-encoding magnetic field gradient are applied at the timing of the readout magnetic field gradient reversing in polarity. First to M-th echoes e1 to eM are sampled at the timing of their refocusing orderly, and imaging data items F(n,1), etc., and F(n,M) are acquired based on the echoes e1, etc., and eM. This application of pulses is repeated n times (where n ranges from 1 to N) by varying the magnitude of the phase-encoding magnetic field gradient pdn. Thus, imaging data items F(1,1) to F(N,M) to be recorded in k-space are acquired. This imaging technique is referred to as an N-shot M-echo technique. Moreover, numbers n assigned orderly to time-sequential shots shall be referred to as shot numbers. Moreover, numbers assigned orderly to echoes, which time-sequentially refocus after returned in response to a certain shot, shall be referred to as echo numbers.

FIG. 6 illustratively shows trajectories along which imaging data items F(1,1) to F(N,M) in the k-space KS are acquired. Herein, N denotes 4 and M denotes 4.

Assume that the k-space KS is divided in the direction of a phase-encoding axis into the first to N×M-th (16th in FIG. 6) rows. In this case, pulses p2, etc., to pM of a phase-encoding magnetic field gradient pdn are applied so that imaging data F(n,m) to be recorded in the (n+(m−1)N)-th row can be acquired based on the m-th echo returned in response to the n-th shot.

The echo planar imaging (EPI) method is sensitive to a phase error. It is therefore required to detect a phase of an MR signal and correct the phase.

FIG. 7 shows an example of a phase detection pulse sequence.

The phase detection pulse sequence is different from the imaging pulse sequence shown in FIG. 5 in a point that the phase-encoding magnetic field gradient is excluded.

Based on the first phase-detection echo E1 to the M-th phase-detection echo EM that refocus orderly, the first phase-detection data D_{—}1 to the M-th phase-detection data D_M are acquired.

Thereafter, the m-th phase-detection data D_m is Fourier-transformed in order to calculate a complex vector Z(n). Herein, 1≦m≦M and 1≦n≦N are met.

Thereafter, a first-order phase φ1_m represented by the m-th phase-detection data D_m is calculated as follows:

$$φ 1 \_m = arg \{ {\sum }_{n = 1}^{N - 1} ( Z ( n + 1 ) / Z ( n ) ) \} \qquad ( 1 )$$

where arg{ } denotes a function providing an argument of a complex. The first-order phase is corrected according to the following expression:

Zcor1(n)=Z(n)·exp{−i·φ1_{—}m·(n−1)} (2)

where exp{ } denotes an exponential function.

Thereafter, a 0-order phase φ0_m represented by the m-th phase detection data D_m is calculated as follows:

$$φ 0 \_m = arg \{ {\sum }_{n = 1}^{N} Zcor1 ( n ) \} \qquad ( 3 )$$

The first-order phase φ1_m and 0-order phase φ0_m are employed in imaging data correction and other various kinds of processing.

Moreover, a 0-order phase difference Δφ0_m between the 0-order phase φ0_m represented by the m-th phase detection data D_m and the 0-order phase φ0_m+1 represented by the (m+1)-th phase detection data D_m+1 is calculated as expressed below.

Δφ0_{—}m=φ0_{—}m+1−φ0_{—}m (5)

The 0-order phase difference Δφ0_m is also employed in various kinds of processing.

FIG. 8 is a conceptual diagram showing phases of complex vectors Z(n), Zcor1(n), and Zcor(n).

By correcting a first-order phase and a 0-order phase, the adverse effect of a phase error on the first to M-th phase detection data items D_{—}1 to D_M can be nullified.

As long as the 0-order phase φ0_m provided as the expression (3) is used in combination with the first-order phase φ1_m provided as the expression (1) in order to correct the first-order phase and 0-order phase successively, no problem occurs.

However, when the 0-order phase φ0_m provided as the expression (3) is solely used for correction or the like, there arises the problem that the result of correction is incorrect. This is because the 0-order phase φ0_m provided as the expression (3) is exactly the phase of the complex vector Z(1) at the first sampling point but does not represent any of the 0-order phases of the complex vectors Z(1) to Z(N) at the first to N-th sampling points.

Moreover, according to the echo planar imaging (EPI) method or gradient and spin echo (GRASE) method, the polarity of a readout magnetic field gradient pulse applied to provide the m-th phase detection data D_m is opposite to the polarity of a readout magnetic field gradient pulse applied to provide the (m+1)-th phase detection data D_m+1. In this case, the 0-order phase difference Δφ0_m calculated according to the expression (5) does not, as shown in FIG. 9, represent a correct 0-order phase difference. Referring to FIG. 9, “readout magnetic field gradient pulses of positive polarity” refer to the odd-numbered readout magnetic field gradient pulses r1, r3, etc. shown in FIG. 7. “Readout magnetic field gradient pulses of negative polarity refer to the even-numbered readout magnetic field gradient pulses r2, r4, etc. shown in FIG. 7.

### SUMMARY OF THE INVENTION

Therefore, the first object of the present invention is to provide a 0-order phase detecting method and a magnetic resonance imaging (MRI) system capable of detecting a 0-order phase that correctly represents any of the 0-order phases of complex vectors Z(1) to Z(N) at the first to N-th sampling points.

The second object of the present invention is to provide a 0-order phase detecting method and a magnetic resonance imaging (MRI) system capable of calculating a correct 0-order phase difference even when a readout magnetic field gradient applied relative to consecutive phase detection echoes reverses in polarity.

According to the first aspect of the present invention, there is provided a 0-order phase detecting method for detecting a 0-order phase φ0. Herein, phase detection data is acquired based on a phase detection echo that is refocused according to a phase detection pulse sequence that does not include phase-encoding magnetic field gradient pulses unlike an imaging pulse sequence. The phase detection data is Fourier-transformed to calculate a complex vector Z(n) at the n-th sampling point which is expressed as follows:

Z(n)=x(n)+i·y(n) (6)

A composite vector Zsum is calculated according to the following expression:

$$Zsum = {\sum }_{n = 1}^{N} \{ x ( n ) \} + i \cdot {\sum }_{n = 1}^{N} \{ y ( n ) \} \qquad ( 7 )$$

A 0-order phase φ0 is detected using the composite vector Zsum as expressed below.

φ0=arg{Zsum} (8)

In the 0-order phase detecting method according to the first aspect, the phase of the composite vector Zsum calculated using the complex vectors Z(1) to Z(N) at the first to N-th sampling points is adopted as a 0-order phase of an MR signal. Compared with a case where the phase of the complex vector Z(1) at the first sampling point is adopted as a 0-order phase of an MR signal as it is according to the related art, a 0-order phase correctly representing any of the 0-order phases of the complex vectors Z(1) to Z(N) at the first to N-th sampling points can be detected.

According to the second aspect of the present invention, in the aforesaid 0-order phase detecting method, assuming that a composite vector calculated based on phase detection echoes that refocus during the first time interval is Zsum_{—}1 and a composite vector calculated based on phase detection echoes that refocus during the second time interval is Zsum_{—}2, a 0-order phase difference Δφ0 is calculated as expressed below.

Δφ0=arg{Zsum_{—}1}−arg{Zsum_{—}2} (9)

In the 0-order phase detecting method according to the second aspect, the difference between the phase arg{Zsum_{—}1} of the composite vector Zsum calculated using complex vectors acquired based on first phase detection echoes and the phase arg{Zsum_{—}2} of the composite vector Zsum calculated using complex vectors acquired based on second phase detection echoes is adopted as a 0-order phase difference Δφ0 exhibited by an MR signal. Even when the polarity of a readout magnetic field gradient pulse applied relative to the first phase detection echoes is opposite to the phase of a readout magnetic field gradient pulse applied relative to the second phase detection echoes, a correct 0-order phase difference can be calculated.

According to the third aspect of the present invention, in the aforesaid 0-order phase detecting method, assuming that a composite vector calculated based on phase detection echoes that refocus during the first time interval is Zsum_{—}1 and a composite vector calculated based on phase detection echoes that refocus during the second time interval is Zsum_{—}2, a 0-order phase difference Δφ0 is calculated as expressed below.

Δφ0=arg{Zsum_{—}1/Zsum_{—}2} (10)

The 0-order phase detecting method according to the third aspect is equivalent to the 0-order phase detecting method according to the second aspect, whereby a correct 0-order phase difference can be calculated.

According to the fourth aspect of the present invention, in the aforesaid 0-order phase detecting method, the imaging pulse sequence is intended to refocus echoes by reversing in polarity a readout magnetic field gradient.

In the 0-order phase detecting method according to the fourth aspect, a pulse sequence is intended to refocus echoes by reversing in polarity a readout magnetic field gradient. Since the pulse sequence is sensitive to a phase error, it is essential to correctly detect a 0-order phase and a 0-order phase difference.

According to the fifth aspect of the present invention, in the aforesaid 0-order phase detecting method, the imaging pulse sequence is adapted to the EPI method or GRASE method.

In the 0-order phase detecting method according to the fifth aspect, the pulse sequence is adapted to the EPI method or GRASE method. Since the pulse sequence is sensitive to a phase error, it is essential to correctly detect a 0-order phase and a 0-order phase difference.

According to the sixth aspect of the present invention, in the aforesaid 0-order phase detecting method, the phase detection echoes refocusing during the first time interval and the phase detection echoes refocusing during the second time interval are consecutive echoes.

In the 0-order phase detecting method according to the sixth aspect, since consecutive echoes are handled, the readout magnetic field gradient reverses in polarity. Nevertheless, a 0-order phase and a 0-order phase difference can be correctly detected.

According to the seventh aspect of the present invention, in the aforesaid 0-order phase detecting method, phase detection data is acquired using a phase detection pulse sequence during a reference scan different from a scan during which imaging data is acquired using an imaging pulse sequence.

In the 0-order phase detecting method according to the seventh aspect, phase detection data is acquired during a reference scan different from a scan during which an imaging pulse sequence is employed. Data acquisition is achieved with little temporal restriction.

According to the eighth aspect of the present invention, in the aforesaid 0-order phase detecting method, pulses are applied according to a phase detection pulse sequence before they are according to an imaging pulse sequence.

In the 0-order phase detecting method according to the eight aspect, pulses are applied according to a phase detection pulse sequence before they are according to an imaging pulse sequence. Therefore, a 0-order phase and a 0-order phase difference detected using the phase detection pulse sequence can be utilized during data acquisition that is performed using the imaging pulse sequence.

According to the ninth aspect of the present invention, there is provided a magnetic resonance imaging (MRI) system consisting mainly of an radio-frequency (RF) pulse transmitting means, a gradient pulse applying means, an MR signal receiving means, a phase detection data acquiring means, a Fourier transforming means, and a 0-order phase calculating means. The phase detection data acquiring means controls the RF pulse transmitting means, gradient pulse applying means, and MR signal receiving means. The phase detection data acquiring means acquires phase detection data on the basis of phase detection echoes received using a pulse sequence that does not unlike an imaging pulse sequence include a phase-encoding magnetic field gradient. The Fourier transforming means Fourier-transforms the phase detection data to calculate a complex vector. Assuming that the complex vector Z(n) at the n-th sampling point is expressed as follows:

Z(n)=x(n)+i·y(n) (6)

a composite vector Zsum is calculated according to the following expression:

$$Zsum = {\sum }_{n = 1}^{N} \{ x ( n ) \} + i \cdot {\sum }_{n = 1}^{N} \{ y ( n ) \} \qquad ( 7 )$$

A 0-order phase φ0 is detected using the composite vector Zsum as expressed below.

φ0=arg{Zsum} (8)

In the MRI system according to the ninth aspect, preferably, the 0-order phase detecting method according to the first aspect is implemented.

According to the tenth aspect of the present invention, the aforesaid MRI system further includes a 0-order phase difference calculating means. Assuming that a composite vector calculated using phase detection echoes that refocus during the first time interval is Zsum_{—}1 and a composite vector calculated using phase detection echoes that refocus during the second time interval is Zsum_{—}2, the 0-order phase difference calculating means calculates a 0-order phase difference Δφ0 as follows:

Δφ0=arg{Zsum_{—}1}−arg{Zsum_{—}2} (9)

In the MRI system according to the tenth aspect, preferably, the 0-order phase detecting method according to the second aspect is implemented.

According to the eleventh aspect of the present invention, the aforesaid MRI system further includes a 0-order phase difference calculating means. Assuming that a composite vector calculated using phase detection echoes that refocus during the first time interval is Zsum_{—}1 and a composite vector calculated using phase detection echoes that refocus during the second time interval is Zsum_{—}2, the 0-order phase difference calculating means calculates a 0-order phase difference Δφ0 as follows:

Δφ0=arg{Zsum_{—}1/Zsum_{—}2} (10)

In the MRI system according to the eleventh aspect, preferably, the 0-order phase detecting method according to the third aspect is implemented.

According to the twelfth aspect of the present invention, in the aforesaid MRI system, the imaging pulse sequence is intended to refocus echoes by reversing in polarity a readout magnetic field gradient.

In the MRI system according to the twelfth aspect, preferably, the 0-order phase detecting method according to the fourth aspect is implemented.

According to the thirteenth aspect of the present invention, in the aforesaid MRI system, the imaging pulse sequence is adapted to the EPI or GRASE method.

In the MRI system according to the thirteenth aspect, preferably, the 0-order phase detecting method according to the fifth aspect is implemented.

According to the fourteenth aspect of the present invention, in the aforesaid MRI system, the phase detection echoes refocusing during the first time interval and the phase detection echoes refocusing during the second time interval are consecutive echoes.

In the MRI system according to the fourteenth aspect, preferably, the 0-order phase detecting method according to the sixth aspect is implemented.

According to the fifteenth aspect of the present invention, in the aforesaid MRI system, phase detection data is acquired using a phase detection pulse sequence during a reference scan different from a scan during which imaging data is acquired using an imaging pulse sequence.

In the MRI system according to the fifteenth aspect, preferably, the 0-order phase detecting method according to the seventh aspect is implemented.

According to the sixteenth aspect of the present invention, in the aforesaid MRI system, pulses are applied according to a phase detection pulse sequence before they are according to an imaging pulse sequence.

In the MRI system according to the sixteenth aspect, preferably, the 0-order phase detecting method according to the eighth aspect is implemented.

According to the 0-order phase detecting method and MRI system in which the present invention is implemented, the phase of a composite vector calculated using the complex vectors at all sampling points that result from Fourier transform of an MR signal is adopted as a 0-order phase. Compared with the related art of adopting the phase of a complex vector at a first sampling point as a 0-order phase, the detected 0-order phase correctly represents the 0-order phase of the MR signal.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram of an MRI system in accordance with an embodiment of the present invention.

FIG. 2 shows a phase detection pulse sequence.

FIG. 3 is a flowchart describing 0-order phase detection in accordance with the embodiment of the present invention.

FIG. 4 is a conceptual diagram showing a 0-order phase and a 0-order phase difference that relate to the present invention.

FIG. 5 shows an imaging pulse sequence adapted to a multi-shot diffusion enhancement EPI method.

FIG. 6 is a conceptual diagram showing trajectories along which imaging data is acquired.

FIG. 7 shows a phase detection pulse sequence.

FIG. 8 is an explanatory diagram concerning first-order phase correction and 0-order phase correction that are performed conventionally.

FIG. 9 is a conceptual diagram showing a 0-order phase and a 0-order phase difference that are adopted conventionally.

### DETAILED DESCRIPTION OF THE INVENTION

The present invention will be described in relation to an embodiment shown in drawings.

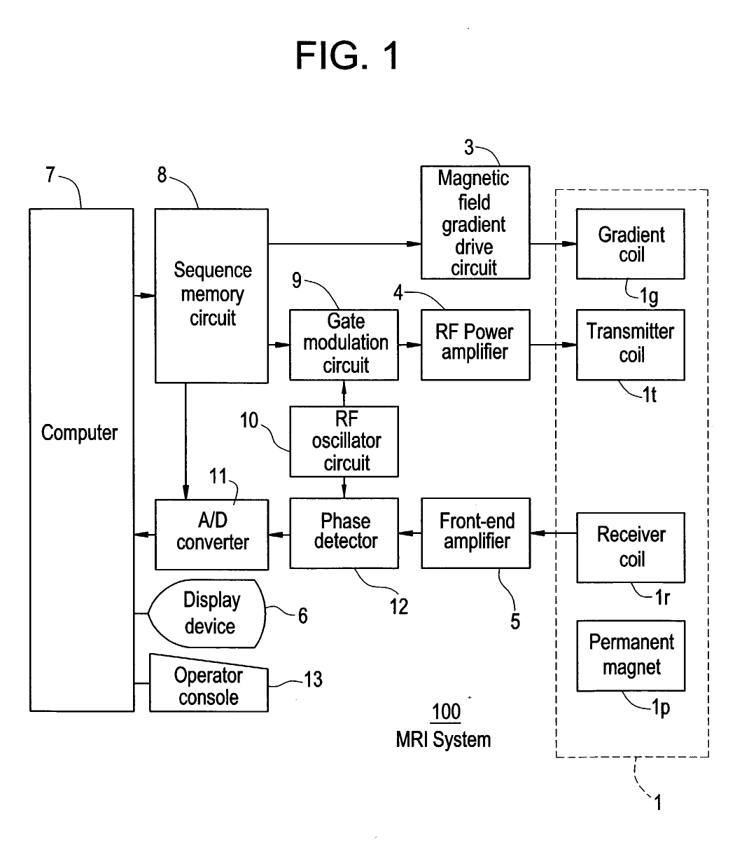

FIG. 1 is a block diagram showing a magnetic resonance imaging (MRI) system according to an embodiment of the present invention.

In the MRI system 100, a magnet assembly 1 has a bore into which a subject is inserted. A permanent magnet 1p, a gradient coil 1g, a transmitter coil 1t, and a receiver coil 1r are disposed around the bore. The permanent magnet 1p applies a constant main magnetic field to the subject. The gradient coil 1g includes x-axis, y-axis, and z-axis coils (a slice-selective axis, a readout axis, and a phase-encoding axis are determined depending on how the coils are combined), and generates magnetic field gradients. The transmitter coil it transmits a radio-frequency (RF) pulse with which the spins of nuclei in the subject are excited. The receiver coil 1r receives an MR signal induced in the subject. The gradient coil 1g, transmitter coil 1t, and receiver coil 1r are connected to a magnetic field gradient drive circuit 3, a radio-frequency (RF) power amplifier 4, and a front-end amplifier 5 respectively. Incidentally, a superconducting magnet may be substituted for the permanent magnet 1p.

A computer 7 composes a pulse sequence and hands it to a sequent memory circuit 8.

The sequence memory circuit 8 holds a pulse sequence. The magnetic field gradient drive circuit 3 operates according to the pulse sequence. The gradient coil 1g included in the magnet assembly 1 generates magnetic field gradients. Moreover, a gate modulation circuit 9 modulates a carrier output signal of a radio-frequency (RF) oscillator circuit 10 into a pulsating signal that has predetermined timing and a predetermined envelope, and applies the pulsating signal as RF pulses to the RF power amplifier 4. The RF power amplifier 4 amplifies the RF pulses and applies the resultant pulses to the transmitter coil it included in the magnet assembly 1.

The front-end amplifier 5 amplifies an MR signal received by the receiver coil 1r included in the magnet assembly 1, and transfers the resultant signal to a phase detector 12. The phase detector 12 uses the carrier output signal of the RF oscillator circuit 10 as a reference signal to detect the phase of the MR signal, and transfers the MR signal to an analog-to-digital (A/D) converter 11. The A/D converter 11 converts the analog MR signal into a digital signal, and transfers the digital signal to the computer 7.

The computer 7 reads the digital signal produced by the A/D converter 11, performs phase detection, phase correction, and image reconstruction, and produces an image. The image is displayed on a display device 6.

Moreover, the computer 7 receives information entered at an operator console 13 and is responsible for overall control.

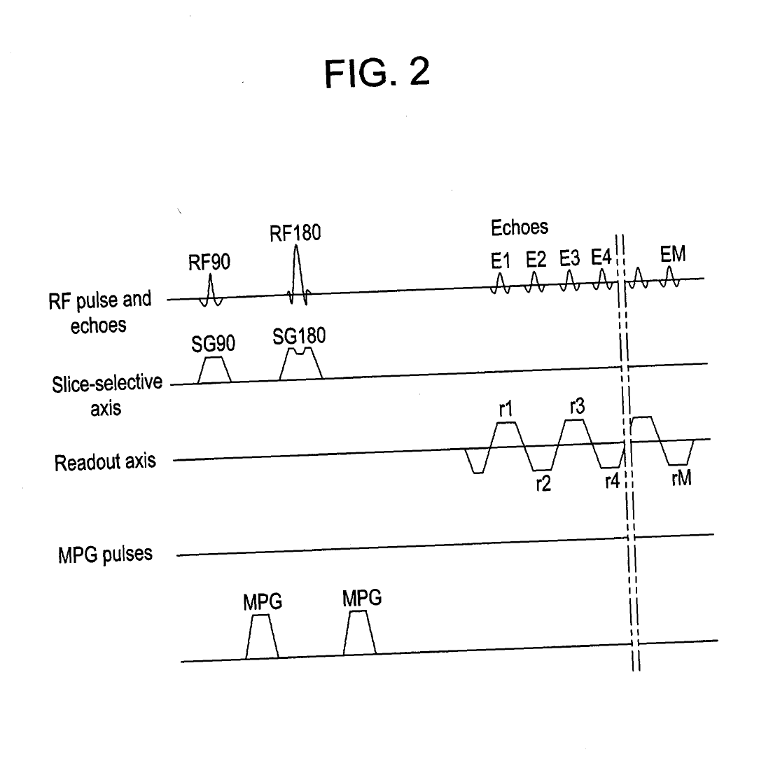

FIG. 2 shows a phase detection pulse sequence employed in the embodiment of the present invention.

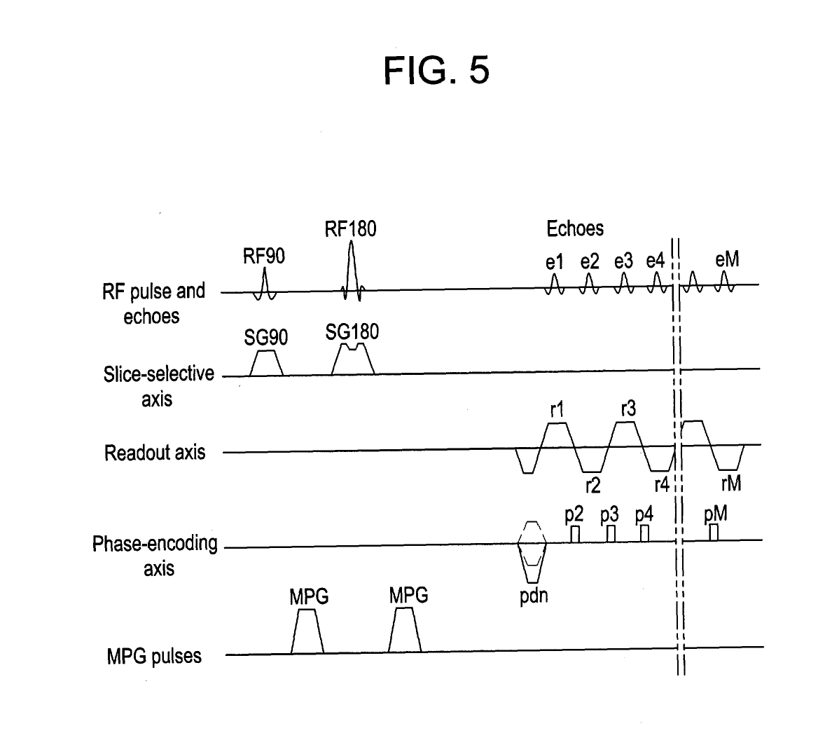

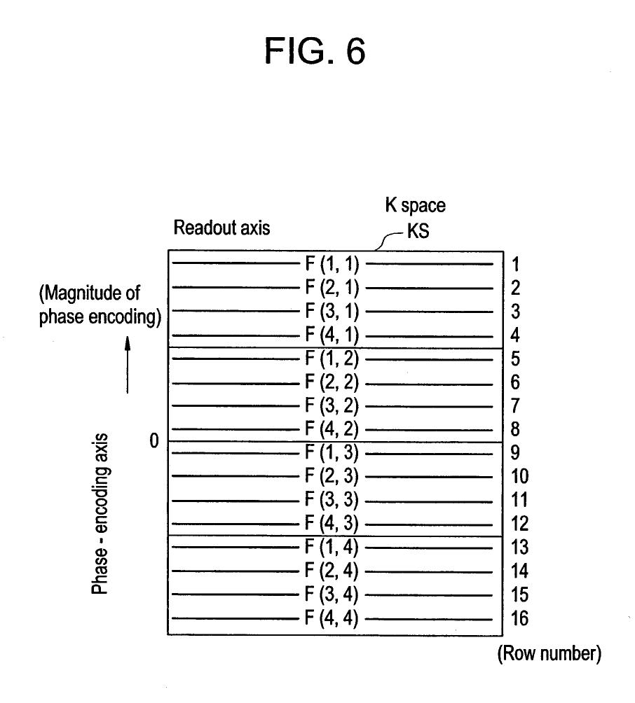

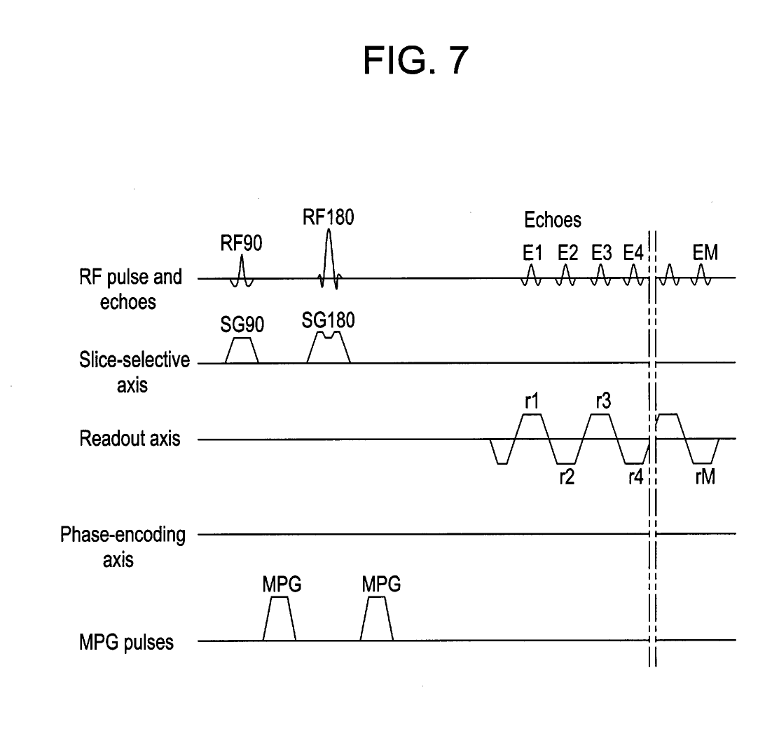

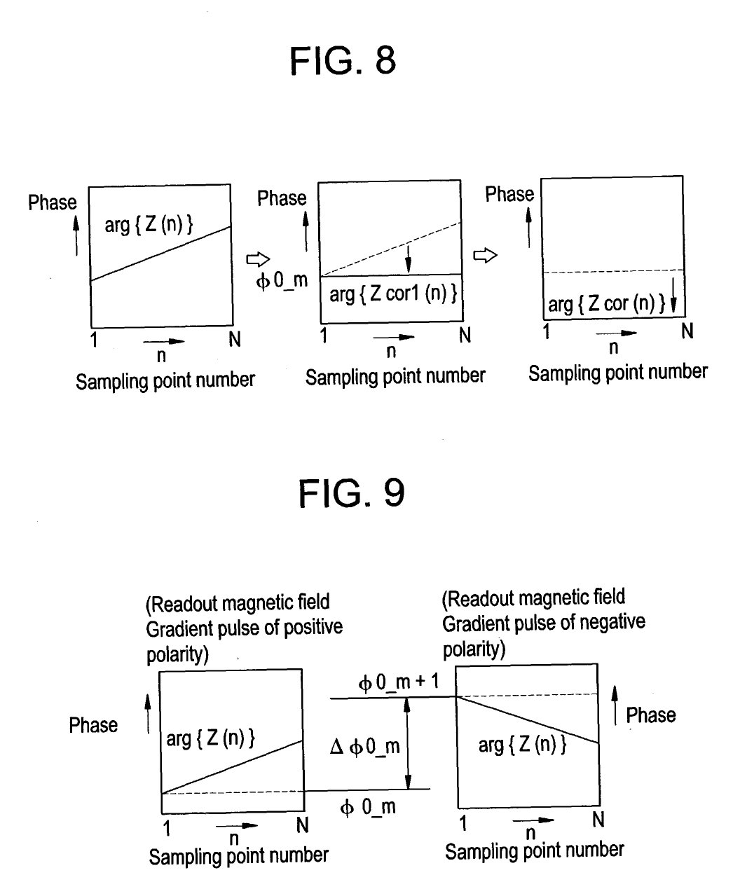

The phase detection pulse sequence does not include a phase-encoding magnetic field gradient unlike an imaging pulse sequence adapted to a multi-shot diffusion enhancement EPI method shown in FIG. 5.

Specifically, an excitation pulse RF90 and a slice-selective magnetic field gradient SG90 are applied, and a motion probing gradient (MPG) pulse MPG is then applied. Thereafter, an inversion RF pulse RF180 and an inversion slice-selective magnetic field gradient SG180 are applied, and another MPG pulse MPG is then applied. Thereafter, pulses r1, etc., and rM of a data acquisition readout magnetic field gradient that alternately reverses in polarity are applied consecutively but no pulse of a phase-encoding magnetic field gradient is applied. First to M-th phase detection data items D_{—}1 to D_M are acquired based on the first to M-th phase detection echoes E1 to EM that refocus orderly.

The phase detection pulse sequence is adopted for a reference scan. After the completion of the reference scan, a scan is performed in order to acquire imaging data according to an imaging pulse sequence.

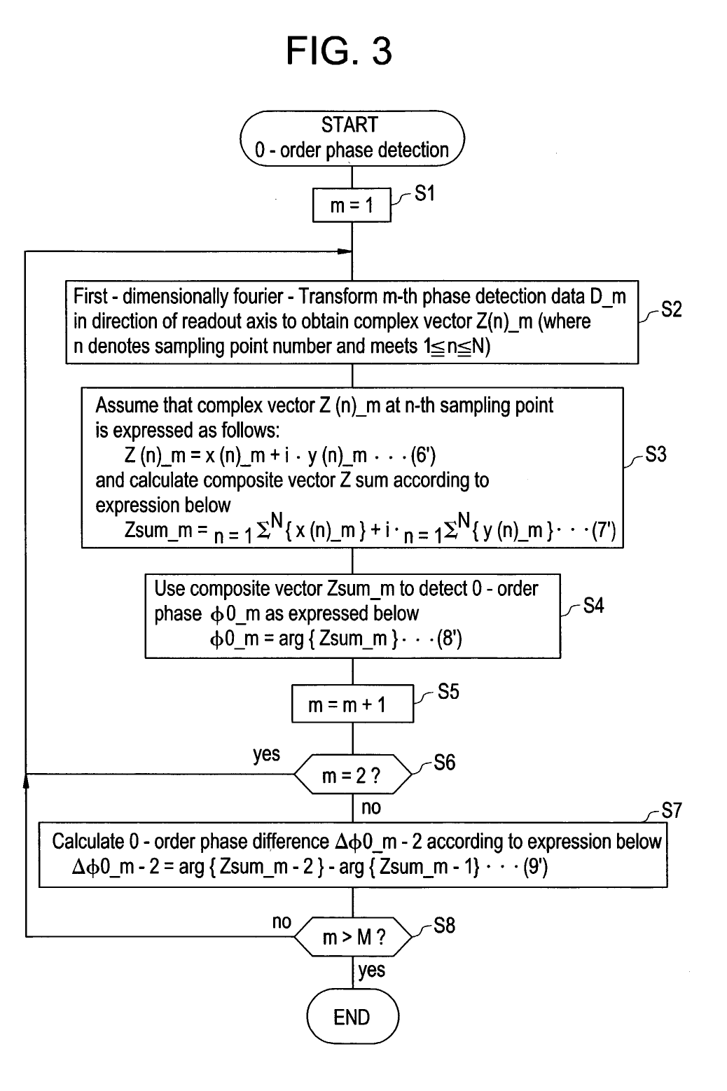

FIG. 3 is a flowchart describing 0-order phase detection performed in the embodiment of the present invention.

At step S1, an echo number counter m is initialized to 1.

At step S2, the m-th phase detection data D_m is first-dimensionally Fourier-transformed in the direction of the readout axis in order to obtain a complex vector Z(n)_m. Herein, n denotes a sampling point number and meets 1≦n≦N.

At step S3, the complex vector Z(n)₁₃ m at the n-th sampling point is expressed as follows:

Z(n)_{—}m=x(n)_{—}m+i·y(n)_{—}m (6′)

A composite vector Zsum_m is calculated as follows:

$$Zsum\_m = {\sum }_{n = 1}^{N} \{ x ( n ) \_m \} + i \cdot {\sum }_{n = 1}^{N} \{ y ( n ) \_m \} \qquad ( {7}^{′} )$$

At step S4, a 0-order phase φ0_m is detected using the composite vector Zsum_m as expressed below.

φ0_m=arg{Zsum_m} (8′)

At step S5, the echo number counter m is incremented by one.

At step S6, if the counter value m equals 2, control is returned to step S2. If the counter value m is equal to or larger than 3, control is passed to step S7.

At step S7, a 0-order phase difference Δφ0_m−2 is calculated as follows:

Δφ0_{—}m−2=arg{Zsum_{—}m−2}−arg{Zsum_{—}m−1} (9′)

At step S8, if the counter value m ranges from 3 to M, control is returned to step S2. If the counter value m equals M+1, the processing is terminated.

Incidentally, at step S7, the 0-order phase difference Δφ0_m−2 may be calculated as follows:

Δφ0_{—}m−2=arg{Zsum_{—}m−1/Zsum_{—}m−2} (10′)

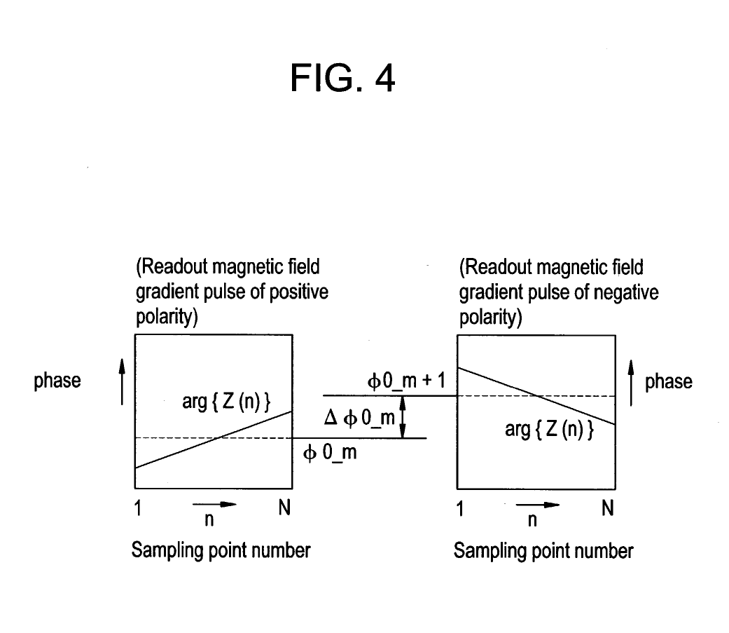

As shown in FIG. 4, the detected 0-order phase φ0 represents any of the 0-order phases of complex vectors at all sampling points. Therefore, even if a readout magnetic field gradient to be applied relative to consecutive phase detection echoes reverses in polarity, the detected 0-order phase difference Δφ0 is correct. Consequently, correction of imaging data and other various kinds of processing in which the 0-order phase difference Δφ0 is employed result in correct data.

Many widely different embodiments of the invention may be constructed without departing from the spirit and the scope of the present invention. It should be understood that the present invention is not limited to the specific embodiments described in the specification, except as defined in the appended claims.

1. A 0-order phase detecting method for detecting a 0-order phase φ0, wherein
phase detection data acquired based on a phase detection echo that is refocused according to a phase detection pulse sequence, which does not include a phase-encoding magnetic field gradient unlike an imaging pulse sequence, is Fourier-transformed in order to calculate a complex vector Z(n) at the n-th sampling point,
assuming that the complex vector Z(n) at the n-th sampling point is expressed as follows:
Z(n)=x(n)+i·y(n)
a composite vector Zsum is calculated according to the expression below; and
$$Zsum = {\sum }_{n = 1}^{N} \{ x ( n ) \} + i$$
a 0-order phase φ0 is detected using the composite vector Zsum as expressed below.
φ0=arg{Zsum}

2. A 0-order phase detecting method according to claim 1, wherein assuming that a composite vector calculated based on phase detection echoes that refocus during the first time interval is Zsum_{—}1 and a composite vector calculated based on phase detection echoes that refocus during the second time interval is Zsum_{—}2, a 0-order phase difference Δφ0 is calculated according to the expression below.
Δφ0=arg{Zsum_{—}1}−arg{Zsum_{—}2}

3. A 0-order phase detecting method according to claim 1, wherein assuming that a composite vector calculated based on phase detection echoes that refocus during the first time interval is Zsum_{—}1 and a composite vector calculated based on phase detection echoes that refocus during the second time interval is Zsum_{—}2, a 0-order phase difference Δφ0 is calculated according to the expression below.
Δφ0=arg{Zsum_{—}1/Zsum_{—}2}

4. A 0-order phase detecting method according to claim 1, wherein the imaging pulse sequence is intended to refocus echoes by reversing in polarity a readout magnetic field gradient.

5. A 0-order phase detecting method according to claim 1, wherein the imaging pulse sequence is adapted to the echo planar imaging (EPI) method or gradient and spin echo (GRASE) imaging method.

6. A 0-order phase detecting method according to claim 1, wherein the phase detection echoes refocusing during the first time interval and the phase detection echoes refocusing during the second time interval are consecutive echoes.

7. A 0-order phase detecting method according to claim 1, wherein phase detection data is acquired using a phase detection pulse sequence during a reference scan different from a scan during which imaging data is acquired using an imaging pulse sequence.

8. A 0-order phase detecting method according to claim 1, wherein pulses are applied according to a phase detection pulse sequence before they are according to an imaging pulse sequence.

9. A magnetic resonance imaging (MRI) system comprising:
a radio-frequency (RF) pulse transmitting device,
a gradient pulse applying device;
an MR signal receiving device,
a phase detection data acquiring device for controlling said RF pulse transmitting device, gradient pulse applying device, and MR signal receiving device, and for acquiring phase detection data on the basis of a phase detection echo received using a phase detection pulse sequence which does not include a phase-encoding magnetic field gradient unlike an imaging pulse sequence;
a Fourier transforming device for Fourier-transforming the/phase detection data to calculate a complex vector; and
a 0-order phase calculating device for assuming that the complex vector Z(n) at the n-th sampling point is expressed as follows:
Z(n)=x(n)+i·y(n)
calculating a composite vector Zsum according to the expression below, and
$$Zsum = {\sum }_{n = 1}^{N} \{ x ( n ) \} + i \cdot {\sum }_{n = 1}^{N} \{ y ( n ) \}$$
detecting a 0-order phase φ0 using the composite vector Zsum as expressed below.
φ0=arg{Zsum}

10. An MRI system according to claim 9, further comprising a 0-order phase difference calculating device which assuming that a composite vector calculated based on phase detection echoes that refocus during the first time interval is Zsum_{—}1 and a composite vector calculated based on phase detection echoes that refocus during the second time interval is Zsum_{—}2, calculates a 0-order phase difference Δφ0 according to the expression below.
Δφ0=arg{Zsum_{—}1}−arg{Zsum_{—}2}

11. An MRI system according to claim 9, further comprising a 0-order phase difference calculating device which assuming that a composite vector calculated based on phase detection echoes that refocus during the first time interval is Zsum_{—}1 and a composite vector calculated based on phase detection echoes that refocus during the second time interval is Zsum_{—}2, calculates a 0-order phase difference Δφ0 according to the expression below.
Δφ0=arg{Zsum_{—}1/Zsum_{—}2}

12. An MRI system according to claim 9, wherein the imaging pulse sequence is intended to refocus echoes by reversing in polarity a readout magnetic field gradient.

13. An MRI system according to claim 9, wherein the imaging pulse sequence is adapted to the echo planar imaging (EPI) method or the gradient and spin echo (GRASE) imaging method.

14. An MRI system according to claim 9, wherein the phase detection echoes refocusing during the first time interval and the phase detection echoes refocusing during the second time interval are consecutive echoes.

15. An MRI system according to claim 9, wherein phase detection data is acquired using a phase detection pulse sequence during a reference scan different from a scan during which imaging data is acquired using an imaging pulse sequence.

16. An MRI system according to claim 9, wherein pulses are applied according to a phase detection pulse sequence before they are according to an imaging pulse sequence.
